(19) United States
(12) Patent Application Publication
(10) Pub. No.: US 2021/0322748 A1
(43) Pub. Date: Oct. 21, 2021

(54) TUBING MARKERS

(71) Applicant: CAREFUSION 303, INC., San Diego, CA (US)

(72) Inventors: Wantjinarjo SUWITO, Irvine, CA (US); Christopher Kazimierz JEDRZEJEWSKI, Alpine, CA (US); Aaron En-Yu WANG, Laguna Hills, CA (US); Corey Mark CHRISTENSEN, Anaheim, CA (US)

(21) Appl. No.: 17/360,589

(22) Filed: Jun. 28, 2021

Related U.S. Application Data
(62) Division of application No. 16/266,877, filed on Feb. 4, 2019.

(51) Int. Cl. A61M 39/10; A61M 5/14

(52) U.S. Cl. CPC A61M 39/10; A61M 2205/6081; A61M 5/14

(57) ABSTRACT

Tube markers for use in infusion sets are provided. An infusion tube includes a tube marker positioned from a tube end at a distance equal to the distance between an external edge and a hard stop of a fluid port of an infusion set component. The tube marker provides an external visual indicator of the fit between the infusion tube and the infusion set component, such as whether there is a gap between the end of the infusion tube and hard stop of the fluid port. The tube marker may be pad printed or laser etched on the tube. The tube marker may also be a tube sleeve over molded or heat shrunk onto the tube. Methods of providing tube markers are also provided.

### CROSS-REFERENCES TO RELATED APPLICATIONS

This application is a divisional of U.S. patent application Ser. No. 16/266,877, entitled “TUBING MARKERS,” filed Feb. 4, 2019, the entirety of which is incorporated herein by reference.

### BACKGROUND

Infusion sets are constructed by joining multiple translucent polymeric tubing segments to multiple polymeric components that are either translucent or opaque. The joints are typically formed by applying a thin layer of solvent or adhesive on one or both of the contacting surfaces and then the two surfaces are brought together. The contacting surface of the tubing can either be at the inner diameter, the outer diameter, or both diameters up to a certain length from one end. The solvent/adhesive is applied either internally, externally, or both. The bonded area, or the bond length for a given diameter, is a critical parameter that if not controlled can cause the infusion sets to leak or separate easily. The bond parameter can vary significantly due to design shortfalls, assembly process shortfalls and process drifts.

Inspecting this critical bond length can be done by visual inspection of the solvent/adhesive wetted area along the contacting surfaces. Another method is inspecting the gap between the tubing end and a hard stop within the component. Both require inspections through translucent components. However, if the component is wavy, textured or opaque, then a visual inspection is difficult to perform, or it cannot be done at all. It is desirable to provide for reliable visual inspections for all infusion set construction regardless of translucency of the components to minimize leaks, separations and variations in total infusion set length.

### SUMMARY

The present disclosure provides tube markers for tubing used in infusion sets to provide an external visual indicator of the fit between an infusion tube and an infusion set component.

In one or more embodiments, a tube for use in an infusion set is provided. The tube includes a body defining a fluid flow pathway, a first tube end and a second tube end. A tube marker is disposed near the first tube end. The tube marker is positioned so that a distance between a leading edge of the tube marker and the outermost surface of the first tube end is substantially equal to a distance between an outermost surface of a fluid port of an infusion set component and a hard stop of the fluid port.

In one or more aspects, the first tube end is a fluid input end configured to be inserted into a fluid outlet port of the infusion set component. In one or more aspects, the first tube end is a fluid output end configured to be inserted into a fluid inlet port of the infusion set component. In one or more aspects, the tube marker includes one or more cylindrical bands. In one or more aspects, the tube marker is pad printed ink, laser etching or a tube sleeve. In one or more aspects, the tube sleeve includes a tapered insertion portion configured to fit inside a portion of the fluid port. In one or more aspects, the tapered insertion portion is configured to displace excess solvent. In one or more aspects, the tube sleeve is configured as a stress relief transition from a stiffness of the infusion set component to a lesser stiffness of the tube.

In one or more aspects, the tube includes a second tube marker disposed near the second tube end, wherein the second tube marker is positioned so that a distance between a leading edge of the second tube marker and the outermost surface of the second tube end is substantially equal to a distance between an outermost surface of a second infusion set component fluid port and a hard stop of the second infusion set component fluid port. In one or more aspects, the first tube end is a fluid input end configured to be inserted into a fluid outlet port of the first infusion set component and the second tube end is a fluid output end configured to be inserted into a fluid inlet port of the second infusion set component. In one or more aspects, the first tube end is a fluid output end configured to be inserted into a fluid inlet port of the first infusion set component and the second tube end is a fluid input end configured to be inserted into a fluid outlet port of the second infusion set component.

In one or more embodiments, an infusion set assembly is provided. The infusion set assembly includes an infusion component and a tube coupled to the infusion component. The tube includes a body defining a fluid flow pathway, a first tube end, a second tube end and a tube marker. The tube marker is positioned near the first tube end so that a distance between a leading edge of the tube marker and the outermost surface of the first tube end is substantially equal to a distance between an external edge and a hard stop of a fluid port of the infusion component.

In one or more aspects, the tube marker includes one of pad printed ink, laser etching and a tube sleeve. In one or more aspects, the tube sleeve includes a tapered insertion portion configured to fit inside a portion of the fluid port, the tapered insertion portion configured to displace a portion of a volume of a bond pocket formed between the tube and the fluid port and a stop portion configured to prevent insertion of the stop portion inside the fluid port. In one or more aspects, the first tube end is one of a fluid input end inserted into a fluid outlet port of the infusion component and a fluid output end inserted into a fluid inlet port of the infusion component. In one or more aspects, the tube marker includes at least one cylindrical band. In one or more aspects, the infusion set assembly includes a second infusion component and a second tube marker disposed near the second tube end. The second tube marker is positioned so that a distance between a leading edge of the second tube marker and the outermost surface of the second tube end is substantially equal to a distance between an external edge and a hard stop of a fluid port of the second infusion component.

Additional features and advantages of the disclosure will be set forth in the description below and, in part, will be apparent from the description or may be learned by practice of the disclosure. The objectives and other advantages of the disclosure will be realized and attained by the structure particularly pointed out in the written description and claims hereof as well as the appended drawings.

It is to be understood that both the foregoing general description and the following detailed description are exemplary and explanatory and are intended to provide further explanation of the disclosure as claimed.

### BRIEF DESCRIPTION OF THE DRAWINGS

The accompanying drawings, which are included to provide further understanding of the disclosure and are incorporated in and constitute a part of this specification, illustrate embodiments of the disclosure and together with the description serve to explain the principles of the disclosure.

FIG. 1 depicts a schematic view of a typical assembled infusion set.

FIG. 2 depicts a schematic view of a typical infusion set component and tubing prior to assembly.

FIG. 3 depicts a cross-section view of the infusion set component of FIG. 2.

FIG. 4 depicts a schematic view of the infusion set component and tubing of FIG. 2 after assembly.

FIG. 5 depicts a cross-section view of the infusion set component and tubing of FIG. 4.

FIG. 6 depicts a schematic view of one or more embodiments of an assembled infusion set component and tubing with markers.

FIG. 7 depicts a cross-sectional view of the infusion set component of FIG. 6.

FIG. 8 depicts a schematic view of one or more embodiments of a tubing segment with markers.

FIG. 9 depicts a cross-sectional view of a typical infusion set component joined with a tubing segment.

FIG. 10 depicts a schematic view of one or more embodiments of a tubing segment with a sleeve.

FIG. 11 depicts a cross-sectional view of one or more embodiments of an infusion set component joined with the tubing segment of FIG. 10.

### DETAILED DESCRIPTION

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions are provided in regard to certain aspects as non-limiting examples. However, it will be apparent to those skilled in the art that the subject technology may be practiced without these specific details. In some instances, well-known structures and components are shown in block diagram form in order to avoid obscuring the concepts of the subject technology.

It is to be understood that the present disclosure includes examples of the subject technology and does not limit the scope of the appended claims. Various aspects of the subject technology will now be disclosed according to particular but non-limiting examples. Various embodiments described in the present disclosure may be carried out in different ways and variations, and in accordance with a desired application or implementation.

Infusion sets may be formed from any combination of infusion components and tubing. Typically, the infusion components and tubing are disposable products that are used once and then discarded. The infusion components and tubing may be formed from any suitable material (e.g., plastic, silicone, rubber). An issue in manufacturing infusion sets is consistently joining the tubing and the infusion components to obtain a secure and/or leak free joint with desired fluid flow. The joint may be formed at either the inner or outer diameter of the tubing. In some cases, the joint may be formed both by bonding inner and outer diameters of the tubing to a component for additional joint strength.

Prior approaches provide a partial solution by making the infusion components translucent. However, even inspecting through a translucent body can be challenging when the outer surface is not smooth or additional layers of material are present, such as a textured gripping surface. In some cases, the infusion components are made opaque and with different colors to indicate a fluid flow direction (e.g., a check valve). Since seeing through opaque material is difficult to impossible, another prior approach is that the joint is typically made at the inner diameter of the tubing. This restricts fluid flow due to a smaller orifice than the tubing inner diameter at the joint. Additionally, applying solvent/adhesive on the inside of the tubing has a higher propensity for occlusions, because the solvent/adhesive is pushed in as the tube is inserted to form a joint.

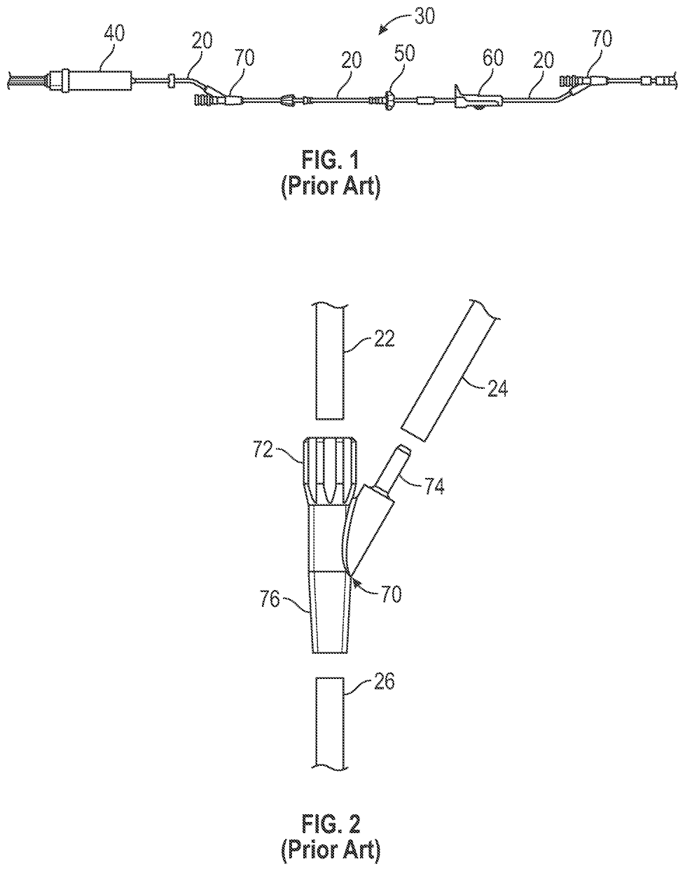

As shown in FIG. 1, a typical infusion set 30 may include a drip chamber 40, a check valve 50, a roller clamp 60 and Y-junctions 70, all connected together by tubing 20. A typical infusion set 30 can include additional infusion components and can be formed of any combination of components and the tubing 20.

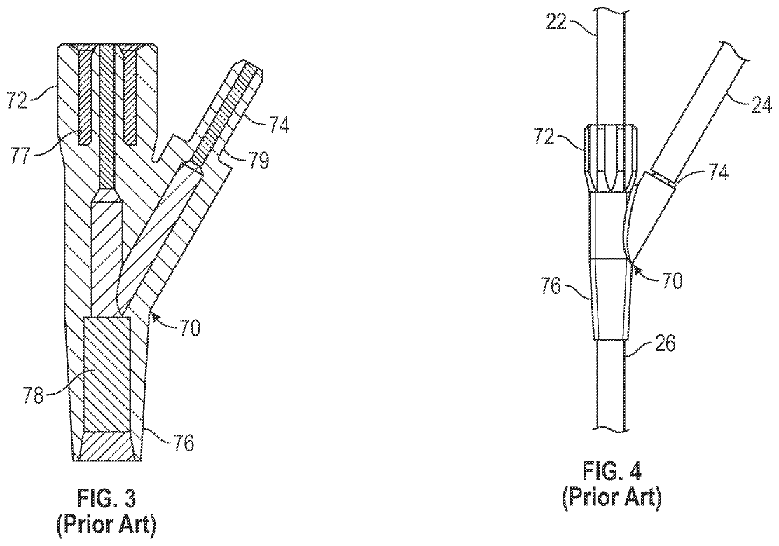

As shown in FIG. 2, a typical Y-junction 70 has inlet ports 72, 74 and an outlet port 76, where each inlet port 72, 74 is to be connected to an inlet tube 22, 24 and the outlet port 76 is to be connected to an outlet tube 26. As shown in FIG. 3, the inlet port 72 has an internal joint 77 where the inlet tube 22 is to be inserted into the inlet port 72. Similarly, the outlet port 76 has an internal joint 78 where the outlet tube 26 is to be inserted into the outlet port 76. By contrast, the inlet port 74 has an external joint 79 where the inlet tube 24 is to be slid onto and over the inlet port 74.

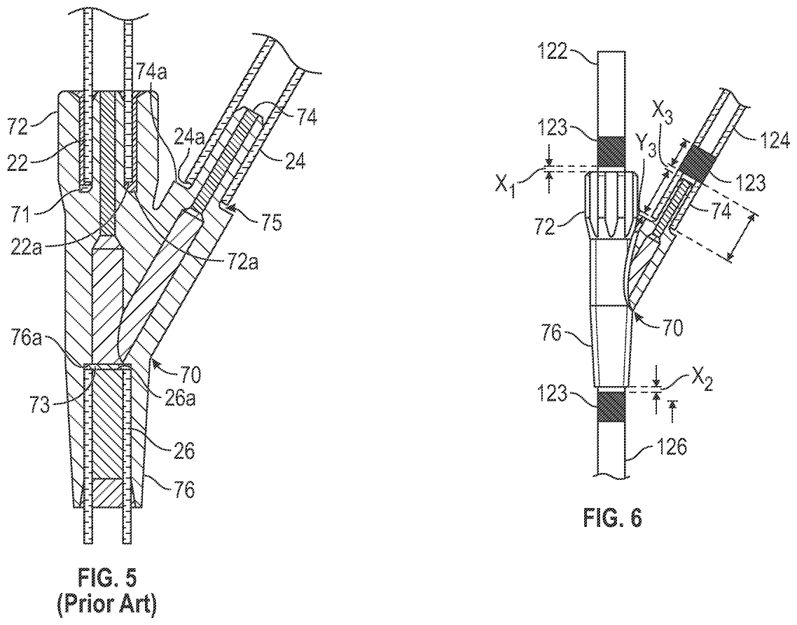

FIGS. 4 and 5 show the Y-junction 70 and the inlet/outlet tubes 22, 24, 26 after assembly. An incomplete joining of the inlet tube 22 with the inlet port 72 leaves a gap 71 between an end surface 22a of the inlet tube 22 and an engagement surface 72a of the inlet port 72. Similarly, an incomplete joining of the outlet tube 26 with the outlet port 76 leaves a gap 73 between an end surface 26a of the outlet tube 26 and an engagement surface 76a of the outlet port 76. Further, an incomplete joining of the inlet tube 24 with the inlet port 74 leaves a gap 75 between an end surface 24a of the inlet tube 24 and an engagement surface 74a of the inlet port 74. As discussed above, the gaps 71, 73, 75 increase the likelihood that the respective internal/external joints 77, 78, 79 will fail, either by leaking or by separation of the inlet/outlet tubes 22, 24, 26 from the Y-junction 70.

Unobstructed visual inspections on sharp edges with good contrast lend themselves for good repeatability and reproducibility (R&R). This is more so for automated computer vision inspections. An acceptable gage R&R is required for measuring critical parameters, such as the case for bond joint area/length. This critical parameter is currently measured by measuring the gap between the tubing end and the insertion hard stop within the component. This method has been found difficult to do in some cases and impossible in others.

By adding sharp contrasting band markers along the tubing, a necessary element to make a good distance measurement is provided. The infusion components are typically injection molded with great dimensional precision. The distance between the hard stop and an external edge can generally be deduced from a drawing. Therefore, the distance between the band marker and that external edge can be used to deduce the bond length.

The marker bands or markers may be produced by a pad printing method, laser marking, or any other suitable method, as long as good contrasting markers are produced. The markers may be high contrasting solid bands. The bands may be two or more fragmented blobs at the outer diameter of tubing. The shape may be a band or any other suitable shape that can be easily seen and analyzed with unaided eyes or computer vision, as well as does not block fluid flow inspection. The addition of markers and others on a translucent tubing may be 40% or less on every inch of the tubing surface area.

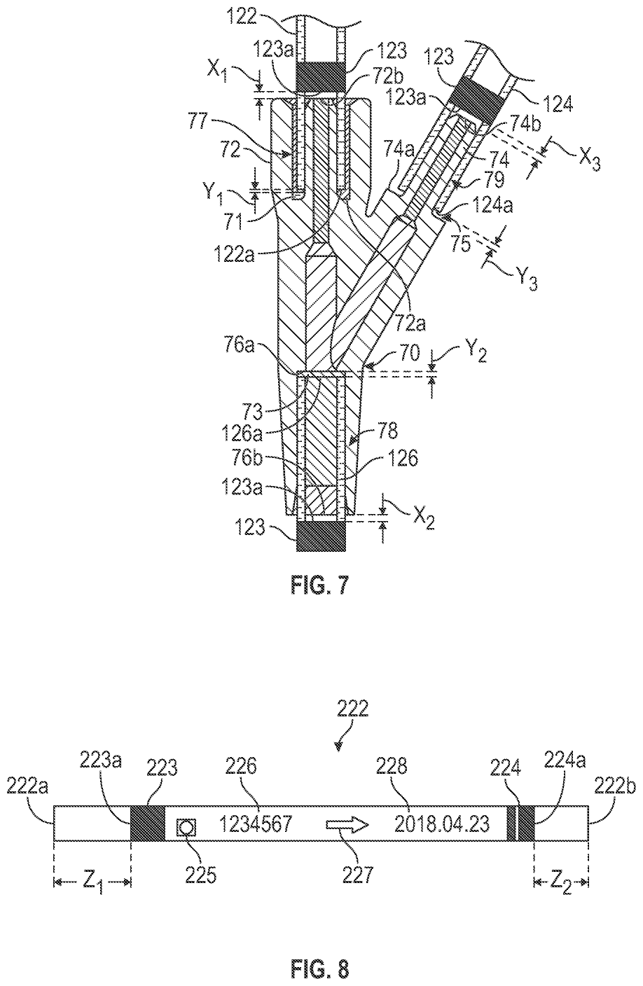

As shown in FIGS. 6 and 7, a Y-joint 70 may be joined together with inlet tubes 122, 124 and outlet tube 126. The inlet tube 122 includes a tube marker 123, which is disposed at a specific position on the inlet tube 122. Thus, when a leading edge 123a of the tube mark 123 is aligned with (e.g., even with) the top surface 72b of the inlet port 122, there is no gap between the end surface 122a of the inlet tube 122 and the engagement surface 72a of the inlet port 72 in the internal joint 77. As another example, if there is a gap 71 between the end surface 122a of the inlet tube 122 and the engagement surface 72a of the inlet port 72, the distance x₁ between the leading edge 123a of the tube mark 123 and the top surface 72b of the inlet port 72 is commensurate or substantially equal to the distance y₁ of gap 71.

Similarly, for the internal joint 78 formed by the outlet tube 126 and the outlet port 76, the outlet tube 126 includes the tube marker 123 disposed at a specific position on the outlet tube 126. Here, when a leading edge 123a of the tube mark 123 is aligned with (e.g., even with) the bottom surface 76b of the outlet port 76, there is no gap between the end surface 126a of the outlet tube 126 and the engagement surface 76a of the outlet port 76. If there is a gap 73 between the end surface 126a of the outlet tube 126 and the engagement surface 76a of the outlet port 76, the distance x₂ between the leading edge 123a of the tube mark 123 and the bottom surface 76b of the outlet port 76 is commensurate or substantially equal to the distance y₂ of gap 73.

For the external joint 79 formed by the inlet tube 124 and the inlet port 74, the inlet tube 124 includes the tube marker 123 disposed at a specific position on the inlet tube 124. When a leading edge 123a of the tube mark 123 is aligned with (e.g., even with) the top surface 74b of the outlet port 74, there is no gap between the end surface 124a of the inlet tube 124 and the engagement surface 74a of the inlet port 74. If there is a gap 75 between the end surface 124a of the inlet tube 124 and the engagement surface 74a of the inlet port 74, the distance x₃ between the leading edge 123a of the tube mark 123 and the top surface 74b of the inlet port 74 is commensurate or substantially equal to the distance y₃ of gap 75.

As shown in FIG. 8, a tube 222 may include markers 223, 224 at both ends for providing visual guidance during joining of the tube 222 to infusion set components. The markers 223, 224 may have different characteristics or looks based on different marker distances from the tube ends. For example, the marker 223 may be a single solid band that is associated with a distance z₁ between the leading edge 223a of the marker 223 and the first tube end 222a. As another example, the marker 224 may be two different sized solid bands that are associated with a distance z₂ between the leading edge 224a of the marker 224 and the second tube end 222b. The tube 222 may also include one or more informational markings, such as a logo 225, a part number/unique device identification 226, a symbol 227 and a date code 228. The informational markings may be disposed between the markers 223, 224 so that the portions of the tube 222 from the leading edge of the markers 223a, 224a to the tube ends 222a, 222b remain clear of markings for ease of use of markers 223, 224. The informational markings may provide relevant information regarding the tubing 222, the infusion set components and/or the infusion set. Thus, the relevant information may be easily discerned from the tubing 222 instead of having to be determined from the associated packaging that is typically discarded.

Another related aspect of the disclosure is determining when and where to add the markers. Infusion set tubing is typically produced using an extrusion method and spooled. The spooled tubing is then cut to desired lengths and the cut tubing is then joined to infusion set components to make an infusion set. The present disclosure provides for a marking tool (e.g., pad printer, laser) to be disposed in a cutting station in which the tubing may be cut to a precise length and markers may be applied at the same cutting and marking machine/station. Thus, no additional process time is incurred by moving materials, loading materials and adding the markers on a different machine/station.

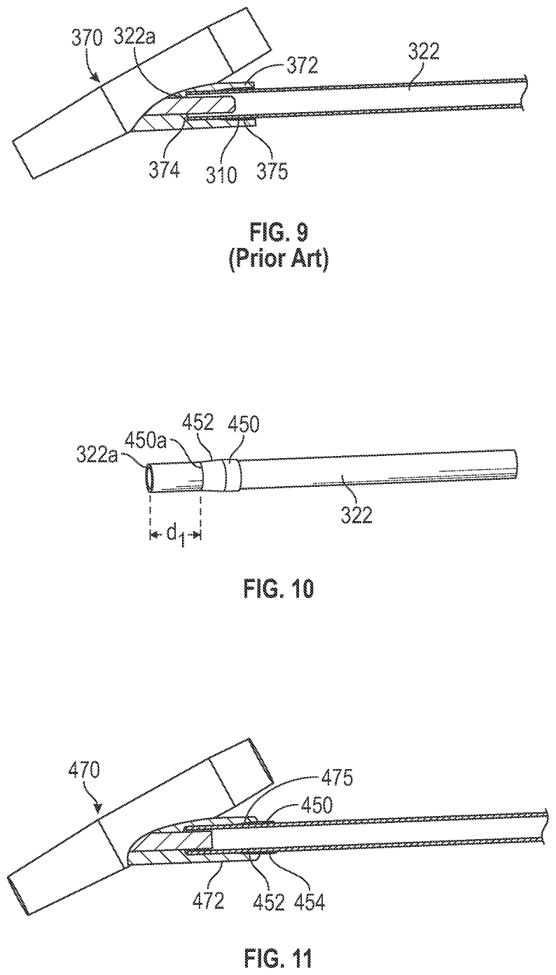

FIG. 9 shows a Y-joint 370 having an inlet port 372 joined or bonded to a typical tube 322 using solvent/adhesive. The inlet port 372 includes a stop member 374 that provides a hard stop when the tube end 322a butts up against the stop member 374 during insertion of the tube 322 into the inlet port 372. A solvent/adhesive pool 310 may form inside a lead-in chamfer of a bond pocket 375. The solvent/adhesive pool 310 may degrade the tube 322 as the solvent/adhesive is curing.

As shown in FIGS. 10 and 11, a tube sleeve 450 may be added onto the tube 322 so that the leading edge 450a of the tube sleeve 450 is a specific distance di from the tube end 322a. The tube sleeve 450 may have a taper such that an insertion portion 452 is sized and shaped to fit inside a part of an infusion set component, such as an inlet port 472 of a Y-junction 470. A stop portion 454 of the tube sleeve 450 may be sized and shaped not to fit into the infusion set component (e.g., inlet port 472), thus preventing a portion of the tube sleeve from sliding into the inlet port 472 in this example. The tube sleeve 450 may eliminate the need to have a mechanical stop inside the infusion set component (e.g., stop member 374 of Y-junction 370), while ensuring that the tube 322 is inserted the correct distance into the inlet port 472.

The tube sleeve 450 may be connected to tube 322 in any suitable manner, such as over molding or heat shrinking into place. The tube sleeve 450 may provide a stress relief transition from a stiff plastic component (e.g., Y-junction 470) to a softer tubing (e.g., tube 322). The tube sleeve 450 may also provide kink resistance to the tube 322 when the tube is confined within packaging. The insertion portion 452 the tube sleeve 450 may further provide for displacement of a portion of the volume of the bond pocket 475, thus preventing the pooling of excess solvent/adhesive.

It is understood that any specific order or hierarchy of blocks in the methods of processes disclosed is an illustration of example approaches. Based upon design or implementation preferences, it is understood that the specific order or hierarchy of blocks in the processes may be rearranged, or that all illustrated blocks be performed. In some implementations, any of the blocks may be performed simultaneously.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

As used herein, the phrase “at least one of” preceding a series of items, with the term “or” to separate any of the items, modifies the list as a whole, rather than each item of the list. The phrase “at least one of” does not require selection of at least one item; rather, the phrase allows a meaning that includes at least one of any one of the items, and/or at least one of any combination of the items, and/or at least one of each of the items. By way of example, the phrase “at least one of A, B, or C” may refer to: only A, only B, or only C; or any combination of A, B, and C.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

It is understood that the specific order or hierarchy of steps, operations or processes disclosed is an illustration of exemplary approaches. Based upon design preferences, it is understood that the specific order or hierarchy of steps, operations or processes may be rearranged. Some of the steps, operations or processes may be performed simultaneously. Some or all of the steps, operations, or processes may be performed automatically, without the intervention of a user. The accompanying method claims, if any, present elements of the various steps, operations or processes in a sample order, and are not meant to be limited to the specific order or hierarchy presented.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112 (f) unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but are to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

1. A tube for use in an infusion set, the tube comprising:
a body defining a fluid flow pathway;
a first tube end;
a second tube end; and
a tube marker disposed near the first tube end, the tube marker comprising one or more cylindrical bands, wherein the tube marker is positioned so that a distance between a leading edge of the tube marker and the outermost surface of the first tube end is substantially equal to a distance between an outermost surface of a fluid port of an infusion set component and a hard stop of the fluid port.

2. The tube of claim 1, wherein the first tube end is a fluid input end configured to be inserted into a fluid outlet port of the infusion set component.

3. The tube of claim 1, wherein the first tube end is a fluid output end configured to be inserted into a fluid inlet port of the infusion set component.

4. The tube of claim 1, wherein the tube marker is printed ink.

5. The tube of claim 1, wherein the tube marker is laser etched.

6. The tube of claim 1, further comprising a second tube marker disposed near the second tube end, wherein the second tube marker is positioned so that a distance between a leading edge of the second tube marker and the outermost surface of the second tube end is substantially equal to a distance between an outermost surface of a second infusion set component fluid port and a hard stop of the second infusion set component fluid port.

7. The tube of claim 6, wherein the first tube end is a fluid input end configured to be inserted into a fluid outlet port of the first infusion set component and the second tube end is a fluid output end configured to be inserted into a fluid inlet port of the second infusion set component.

8. The tube of claim 6, wherein the first tube end is a fluid output end configured to be inserted into a fluid inlet port of the first infusion set component and the second tube end is a fluid input end configured to be inserted into a fluid outlet port of the second infusion set component.

9. An infusion set assembly, comprising:
an infusion component; and
a tube coupled to the infusion component, the tube comprising:
a body defining a fluid flow pathway;
a first tube end;
a second tube end; and
a tube marker disposed near the first tube end, the tube marker comprising one or more cylindrical bands, wherein the tube marker is positioned so that a distance between a leading edge of the tube marker and the outermost surface of the first tube end is substantially equal to a distance between an external edge and a hard stop of a fluid port of the infusion component.

10. The tube of claim 9, wherein the tube marker is printed ink.

11. The tube of claim 9, wherein the tube marker is laser etched.

12. The infusion set assembly of claim 9, wherein the first tube end is a fluid input end inserted into a fluid outlet port of the infusion component.

13. The infusion set assembly of claim 9, wherein the first tube end is a fluid output end inserted into a fluid inlet port of the infusion component.

14. The infusion set assembly of claim 9, further comprising:
a second infusion component; and
a second tube marker disposed near the second tube end, wherein the second tube marker is positioned so that a distance between a leading edge of the second tube marker and the outermost surface of the second tube end is substantially equal to a distance between an external edge and a hard stop of a fluid port of the second infusion component.
